(19) United States
(12) Patent Application Publication
(10) Pub. No.: US 2016/0374281 A1
(43) Pub. Date: Dec. 29, 2016

(54) Ancho Chile Pepper Variety

(71) Applicant: Edward N. Curry, Pearce, AZ (US)

(72) Inventor: Edward N. Curry, Pearce, AZ (US)

(21) Appl. No.: 14/750,856

(22) Filed: Jun. 25, 2015

(51) Int. Cl. A01H 5/08

(52) U.S. Cl. CPC A01H 5/08

(57) ABSTRACT

An improved ancho-type chile pepper cultivar that produces fruit (i.e., peppers) that is characterized by rounded shoulders with little or no indentation at the calyx at the top of the fruit in comparison to existing ancho-type chile pepper cultivars. Plants and fruit grown from the seed of the improved cultivar also are described.

### FIELD OF THE INVENTION

The invention relates to a novel variety of ancho-type chile pepper that results in a fruit that has a rounded shoulder with little to no indentation at the calyx compared to other ancho-type varieties in pepper cultivars of the genus Capsicum.

### BACKGROUND OF THE INVENTION

Within the genus Capsicum, the species Capsicum annuum L. cultivars possess a range of pod shapes and colors complemented by varying degrees seed content, skin thickness, and flavor intensity. Each cultivar has a “heat” or pungency that ranges from mild to hot. The pungent active ingredient found in peppers is the aromatic phenol capsaicin, which is produced by oil secreting glands located along the placenta. Thus, pepper varieties having smaller placenta content typically are milder in flavor.

The commercial value of chile peppers lies in the recovery of the flesh (which excludes seeds, skin, and placenta) and is the raw material for several chile-pepper consumer products, and in the seeds, which are sold to agricultural producers of the fruit. Thus, the commercial processing of chile peppers necessarily involves the separation of flesh and seeds from the rest of the fruit (skin and placenta) and from other materials (such as the stem and calyx) that may be collected during harvesting of the pepper.

The term “poblano” is used by U.S. produce managers for all green fruits of the ancho type. Ancho-type chile peppers have a heart-shaped, pointed, thin walled fruit, with the stem attachment to the calyx on the fruit being indented. This indentation can harbor moisture and non-beneficial organisms, such as bacteria or fungus that cause disease.

### SUMMARY OF THE INVENTION

The disclosure herein relates to Capsicum annuum seeds and plants or varieties (“cultivars”) that produce fruit (i.e., peppers) that is characterized rounded shoulders with little or no indentation at the calyx in comparison to existing ancho-type chile pepper cultivars.

As used herein, the terms “little indentation” or “near absence of indentation” are defined as an indentation at the calyx of no more than about 1 millimeter relative to the top of the pepper. In comparison, the typical indentation on an ancho type pepper is at least 3-5 millimeters.

Various other purposes and advantages of the invention will become clear from its description in the specification that follows. Therefore, this invention includes the features hereinafter described in the detailed description of the preferred embodiments, and particularly pointed out in the claims. However, such description discloses only some of the various ways in which the invention may be practiced.

### BRIEF DESCRIPTION OF THE FIGURES

FIG. 1 shows a comparison of the shoulders/calyx region between a typical ancho-type chile variety (right pepper shows indentation) and an ancho cultivar disclosed herein (left pepper with little or no indentation).

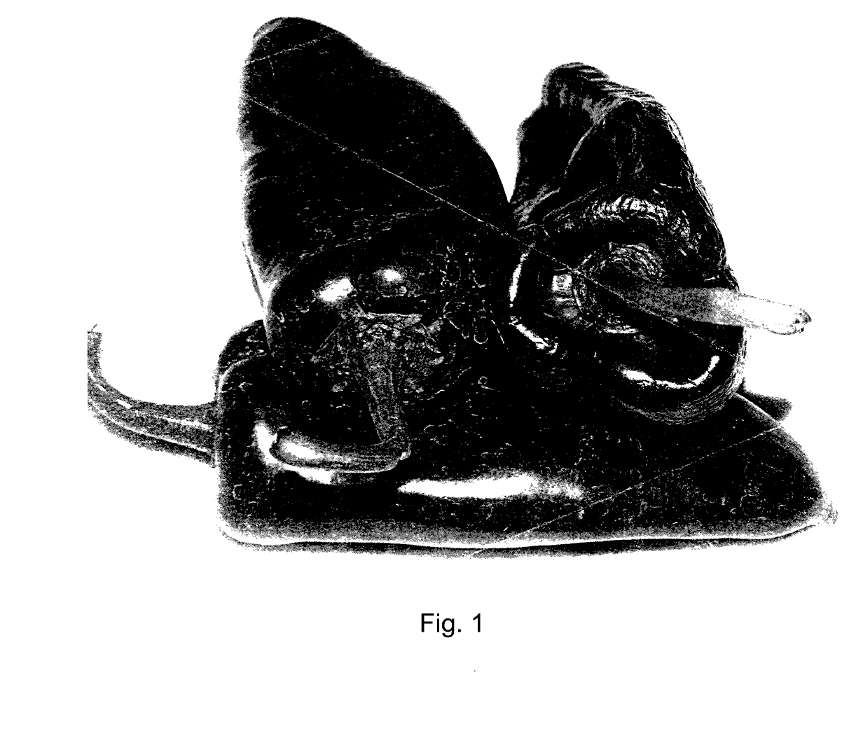

### DETAILED DESCRIPTION OF THE INVENTION

Pedigree Synopsis for Ancho-type Chile Pepper Cultivar Villa 96

The basic parents of the Villa 96/Ancho cultivar are the San Martin Ancho F2 Line and Breeding Line 17 (an ancho-type long green chile pepper). The above Lines were crossed using standard Mendalian genetics and eight generations were grown under typical chile growing conditions in Pearce, Ariz. Field selections were performed based on rounded shoulders and the absence or near absence of detention at the calyx, along with typical ancho-type characteristics, such as short (2.5-3 inches in length), heart-shaped, pointed, and thin walled fruit.

It was discovered that the absence or near absence of an indented calyx substantially reduced bacteria spot (Xanthomonas, common leaf spot) in the improved cultivar in comparison to other ancho-type varieties (such as the parent varieties).

Thus, an ancho-type chile pepper cultivar of the invention has a fruit that is more disease resistant in comparison to existing ancho-type chile pepper cultivars.

Deposit Information

Two thousand five hundred (2500) seeds of Ancho-type Villa 96 Chile cultivar described herein have been placed on deposit with the American Type Culture Collection (ATCC), 10801 University Blvd., Manassas, Va., 20110-2209 under Patent Deposit Designation PTA-122196 on June 3, 2015. This deposit was made in compliance with the Budapest Treaty requirements that the duration of the deposit should be for thirty (30) years from the date of deposit or for five (5) years after the last request for the deposit at the depository or for the enforceable life of a U.S. Patent that matures from this application, whichever is longer. These Capsicum annuum seeds will be replenished should it become non-viable at the depository.

Plants and fruit resulting from the deposited seed have been grown in Southern Arizona and found to exhibit the little or no calyx indentation phenotype and other characteristics described herein.

The ancho-type chile cultivars of the present invention are genetically stable. Nonetheless, the characteristics described herein may be adversely affected by environmental factors (such as high temperatures, low soil fertility, or water stress) and may vary in fruit from plant to plant, while still maintaining a little or no calyx indentation phenotype in comparison to other ancho-type chile varieties.

Various changes in the details and components that have been described may be made by those skilled in the art within the principles and scope of the invention herein described in the specification and defined in the appended claims. Therefore, while the present invention has been shown and described herein in what is believed to be the most practical and preferred embodiments, it is recognized that departures can be made therefrom within the scope of the invention, which is not to be limited to the details disclosed herein but is to be accorded the full scope of the claims so as to embrace any and all equivalent processes and products.

What is claimed is:

1. A Capsicum annuum, ancho-type chile pepper plant from a cultivar of the Villa 96/Ancho line, representative seed of said line having been deposited under ATCC Patent Deposit Designation No. PTA-122196.

2. A seed produced from the plant of claim 1.

3. A fruit resulting from a plant produced from the seed of claim 2.

4. A plant resulting from the seed of claim 2.
